(19) United States
(12) Patent Application Publication
(10) Pub. No.: US 2006/0105423 A1
(43) Pub. Date: May 18, 2006

(54) Microorganisms as carriers of nucleotide sequences coding for cell antigens used for the treatment of tumors

(76) Inventors: Ulf R. Rapp, Wurzburg (DE); Werner Goebel, Gerbrunn (DE); Ivaylo Gentschev, Kist (DE); Joachim Fensterle, Hochberg (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/506,096

(22) PCT Filed: Feb. 13, 2003
(86) PCT No.: PCT/DE03/00471

§ 371 Date: Jun. 11, 2005

(30) Foreign Application Priority Data
Feb. 28, 2002 (DE) 102 08 653.2

(51) Int. Cl. C07K 14/195; C07K 14/245; C07H 21/04; C12P 21/06; C12N 15/74; C12N 1/21

(52) U.S. Cl. 435/69.1; 530/350; 435/252.3; 435/252.33; 435/488; 536/23.2

(57) ABSTRACT

The invention relates to a microorganism with a nucleotide sequence coding for a cell antigen in which the following components are inserted and are expressible: I) a nucleotide sequence coding for at least one epitope of an antigen of a tumor cell and/or a nucleotide sequence for at least one epitope of an antigen that is specific for a tissue cell from which the tumor originates; II) an optional nucleotide sequence coding for a protein that stimulates cells of the immune system; IIIA) a nucleotide sequence for a transport system which makes it possible to express the expression product of components I) and, optionally, II) on the outer surface of the bacterium and/or secrete the expression product of component I) and, optionally, of component II); and/or IIIB) a nucleotide sequence for a protein used for lysing the microorganisms in the cytosol of mammalian cells and for intracellularly releasing plasmids which are contained in the lysed microorganisms; and IV) an activation sequence for expressing one or several of components I) to IIIB), said activation sequence being selected among the group consisting of an activation sequence which is capable of being activated in the microorganism, is tissue-cell-specific but not cell-specific. Each of components I) to IV) can be identically or differently arranged in an individual or multiple manner. Also disclosed are uses of such a microorganism for the production of a medicament.

### FIELD OF THE INVENTION

The invention relates to a microorganism with foreign nucleotide sequences, to the use thereof as a medicament, in particular vaccine, to a plasmid with the foreign nucleotide sequences and a method for the production of such a microorganism.

### BACKGROUND OF THE INVENTION AND PRIOR ART

The main reason for the in most cases lethal consequence of malignant tumor diseases is the inability of the body's defense system to detect and destroy malignant cancer cells. In the industrial countries, cancer diseases belong to the most common diseases with lethal course. In Germany alone, more than 210,000 people die per year because of malignant new formations (source: WHO, figures of 1997), which corresponds to a yearly rate of more than 255 deaths per 100,000 inhabitants.

The basis of this invention are newer findings in the molecular mechanisms leading to malignant deformations. In an early stage already of the cancer formation, there are characteristic changes of the control of cell growth and/or cell differentiation (Pronten, Cancer Surv. 32:5-35, 1998). Essentially involved in these changes are proteins of the signal transduction and the cell cycle control, which were identified in the last years, and all of which are also tumor antigens.

Tumor antigens are roughly divided into three groups (Pardoll, Nat. Med. 4:525-531, 1998): i) tumor-specific neoantigens, which exist in the tumor cell in a mutated and/or over-expressed form, such as EGF-R, HER-2, ii) tumor-specific embryonic antigens, such as members of the MAGE protein family or CEA, iii) tumor-tissue-specific differentiation antigens, such as tyrosinase, Mart-1/Melan-A and gp100.

For the effectiveness of a tumor vaccine, an effective induction of CD8+ T cells is decisive, since tumor cells do in most cases not represent MHC class II molecules, and the intracellularly existing tumor antigens are in most cases MHC class I restringed. For tumor patients, the naturally occurring populations of CD8+, cytotoxic T cells (CTL), are obviously not sufficient to detect and eliminate the tumor cells (Jaffee, Ann. N.Y. Acad. Sci. 886:67-72, 1999). Furthermore, tumor-specific T cells cannot effectively attack the tumor tissue due to various mechanisms (anergy, tolerance, neutralization) (Smyth et al., Nat Immunol 2:293-299, 2001). A successful vaccine must therefore break this anergy or tolerance and induce a sufficient number of activated, specific CTL as well as of specific antibodies. The role of specific antibodies can be seen by the successful use of monoclonal antibodies (mAbs) against tumor antigens of the group (a), such as the already commercially available herceptin, a mAb against HER-2 (Colomer et al., Cancer Invest 19:49-56, 2001).

It is already known that attenuated intracellular bacteria are suitable as vaccine carriers against certain bacterial infections, which in particular can be controlled by a so-called Th1 immune response (Hess and Kaufmann, FEMS Immunology & Medical Microbiology 23:165-173, 1999). This response is characterized by CTL and the presence of specific IFN-g secreting CD4+ T cells (also T helper cells, Th) (Abbas et al., Nature 383:787-793, 1996). Other groups have shown that recombinant bacteria can protect against a heterologous tumor (Medina et al., Eur. J. Immunol. 29:693-699, 1999; Pan et al., Cancer Res. 59:5264-5269, 1999; Woodlock et al., J. Immunother. 22:251-259, 1999; Paglia et al., Blood 92:3172-3176, 1998; Paglia et al., Eur. J. Immunol. 27:1570-1575, 1997; Pan et al., Nat. Med. 1:471-477, 1995; Pan et al., Cancer Res. 55:4776-4779, 1995). In these cases, however, animals were immunized against a surrogate antigen, and then tumor cells expressing this antigen were applied.

These tumor systems cannot however be compared to clinical tumors, since in these models there were no tolerance for the tumor antigen.

A considerable number of different tumor vaccines have already been clinically investigated. Up to now, however, a break-through for the treatment of tumor diseases could not be achieved with any of the tumor vaccines or vaccination methods. In view of this background, there continues to exist an extremely high need of new tumor therapy methods.

It is known in the art to express expression products of nucleic acid sequences introduced into bacteria on the cell membrane of these bacteria, or to have them secreted from these bacteria. The basis of this technique is the Escherichia coli hemolysin system HlyAs representing the prototype of a type I secretion system of gram-negative bacteria. By means of the HlyAs, secretion vectors were developed, which permit an efficient discharge of protein antigens in Salmonella enterica, Yersinia enterocolitica and Vibrio cholerae. Such secretion vectors contain the cDNA of an arbitrary protein antigen coupled to the nucleotide sequence for the HlyA signal peptide, for the hemolysin secretion apparatus, hlyB and hlyD and the hly-specific promoter. By means of this secretion vector, a protein can be expressed on the surface of this bacterium. Such genetically modified bacteria induce as vaccines a considerably higher immune protection than bacteria, in which the protein expressed by the introduced nucleic acid remains inside the cell (Donner et al EP 1015023A; Gentschev et al, Gene, 179:133-140, 1996; Vaccine 19:2621-2618, 2001; Hess et al PNAS 93:1458-1463, 1996). The disadvantage of this system is however that by the use of the hly-specific promoter, the amount of the protein expressed on the exterior surface of the bacterium is extremely small.

A technique for inserting plasmid DNA into mammalian cells by carrier bacteria such as Salmonella and Listeria monocytogenes was developed. Genes contained in these plasmids could also be expressed in the mammalian cells, when they were under the control of a eukaryotic promoter. Plasmids were introduced into Listeria monocytogenes germs, said plasmids containing a nucleotide sequence for an arbitrary antigen under the control of an arbitrary eukaryotic promoter. By introduction of the nucleotide sequences for a specific lysis gene, it was obtained that the Listeria monocytogenes germs dissolve in the cytosol of the antigen-presenting cell and release their plasmids, which leads to a subsequent expression, processing and presentation of the plasmid-coded proteins and clearly increases the immunogenecity of these proteins (Dietrich et al. Nat. Biotechnol. 16:181-185, 1998; Vaccine 19:2506-2512, 2001).

Virulence-attenuated, intracellularly settling bacteria were developed. For instance such variants of Listeria monocytogenes, Salmonella enterica sv. typhimurium and typhi, and Mycobacterium bovis were already used as well-tolerated live vaccines against typhus and tuberculosis. These bacteria, including their attenuated mutants are generally immune-stimulating and can initiate a fair cellular immune response. For instance, L. monocytogenes stimulates to a special extent by the activation of THl-cells the proliferation of cytotoxic T-lymphocytes. These bacteria supply secerned antigens directly into the cytosol of antigen-presenting cells (APC; macrophages and dendritic cells), which in turn express the co-stimulating molecules and cause an efficient stimulation of T cells. The listeriae are in part degraded in phagosomal compartments, and the antigens produced by these carrier bacteria can therefore on the one hand be presented by MHC class II molecules and thus lead to the induction of T helper cells. On the other hand, the listeriae replicate after release from the phagosome in the cytosol of APCs; antigens produced and secerned by these bacteria are therefore preferably presented by the MHC class I pathway, thus CTL responses against these antigens being induced. Further it could be shown that by the interaction of the listeriae with microphages, natural killer cells (NK) and neutrophilic granulocytes, the expression of such cytokines (TNF-alpha, IFN-gamma, IL-2, IL-12; Unanue, Curr. Opin. Immunol., 9:35-43, 1997; Mata and Paterson, J. Immunol. 163:1449-14456, 1999) is induced, for which an antitumoral efficiency was detected. By the administration of L. monocytogenes, which were transduced for the expression of tumor antigens, the growth of experimental tumors could be inhibited antigen-specifically (Pan et al., Nat Med 1:471-477, 1995; Cancer Res. 59:5264-5269, 1999; Voest et al., Natl. Cancer Inst. 87:581-586, 1995; Beatty and Paterson, J. Immunol. 165:5502-5508, 2000).

Virulence-attenuated Salmonella enterica strains, into which nucleotide sequences coding for tumor antigens had been introduced, as tumor antigen-expressing bacterial carriers, could provide after oral administration a specific protection against different experimental tumors (Medina et al., Eur. J. Immunol. 30:768-777, 2000; Zoller and Christ, J. Immunol. 166:3440-34450, 2001; Xiang et al., PNAS 97:5492-5497, 2000).

Recombinant Salmonella strains were also effective as prophylactic vaccines against virus infections (HPV); (Benyacoub et al., Infect Immun 67:3674-3679, 1999) and for the therapeutic treatment of a mouse tumor immortalized by a tumor virus (HPV) (Revaz et al., Virology 279:354-360, 2001).

### TECHNICAL OBJECT OF THE INVENTION

It is the object of the present invention to provide a medicament, which in particular represents in the tumor prophylaxis and tumor therapy an improved vaccine for breaking the immune tolerance with respect to tumors.

### BASIC CONCEPT OF THE INVENTION

For achieving this technical object, the invention teaches a microorganism with a nucleotide sequence coding for a cell antigen, in the genome of which the following components are inserted and are expressible: I) a nucleotide sequence coding for at least one epitope of an antigen or several antigens of a tumor cell and/or a nucleotide sequence for at least one epitope of an antigen or several antigens that is or are specific for a tissue cell from which the tumor originates; II) an optional nucleotide sequence coding for a protein that stimulates cells of the immune system; IIIA) a nucleotide sequence for a transport system, which makes it possible to express the expression product of components I) and, optionally, II) on the outer surface of the bacterium and/or secrete the expression product of component I) and, optionally, of component II); and/or IIIB) a nucleotide sequence for a protein used for lysing the microorganisms in the cytosol of mammalian cells and for intracellularly releasing plasmids, which are contained in the lysed microorganisms; and IV) an activation sequence for expressing one or several of components I) to IIIB), said activation sequence being selected among the group consisting of “an activation sequence, which is capable of being activated in the microorganism, is tissue-cell-specific, but not cell-specific”, and each of components I) to IV) can be identically or differently arranged in an individual or multiple manner, and uses of such a microorganism for the production of a medicament.

Thus, subject matter of the invention are microorganisms, which represent carriers of nucleotide sequences coding for cell antigens, which in turn are expressed or secreted on the outer membrane of the microorganisms, and the use of these microorganisms for breaking the immune tolerance against tumors, and new tumor vaccines that contain microorganisms as carriers of nucleotide sequences coding for cell antigens of normal cells and/or of tumor cells. By the invention, at last an immune reaction directed against the tumor is caused.

In detail, the microorganisms according to the invention contain the following components: I) at least one nucleotide sequence coding for at least one epitope of at least one antigen of at least one cell protein of a tumor cell and/or, optionally, at least one nucleotide sequence for at least one epitope of at least one antigen that is specific for the tissue cell from which the tumor originates; II) optionally, at least one nucleotide sequence for at least one protein that stimulates cells of the immune system; IIIA) at least one nucleotide sequence for a transport system for expressing or secreting the cell antigen coded by component I) on the membrane and for secreting the immune-stimulating protein coded by component; IIIB) optionally, a nucleotide sequence for a lysine lysing the microorganism in the cytosol, so that plasmids, which are contained in the microorganism, are released into the cytosol; IV) at least one nucleotide sequence for an activation sequence that is capable to be activated in the microorganism or activated not cell-specifically, but tumor cell-specifically, tissue cell specifically or function-specifically for expressing components I) and II).

### PREFERRED EMBODIMENTS

In the following, the components of a microorganism according to the invention are described in detail.

Component I).

Component I) represents at least one nucleotide sequence for at least one epitope of at least one antigen of at least one cell protein or at least one oncogenically mutated cell protein of a tumor cell. The oncogenic mutation of the cell protein may have caused a loss or a gain of its original cellular functions. Furthermore, this cell protein can be selected among the group consisting of “receptor molecules or parts thereof, namely extracellular, transmembranic or intracellular parts of the receptors; adhesion molecules or parts thereof, namely extracellular, transmembranic or intracellular parts of the adhesion molecules; proteins of the signal transduction; proteins of the cell cycle control; differentiation proteins; embryonic proteins; and virus-induced proteins”. Such cell antigens perform in the cell the control of the cell growth and of the cell division and are presented on the cell membrane of normal cells, for instance by the MHC class I molecule. In tumor cells, these cell antigens are frequently over-expressed or specifically mutated. Such mutations can have function limitations of oncogene suppressors or the activation of proto-oncogenes to oncogenes as a consequence and can be involved alone or commonly with over-expressions in the tumor growth. Such cell antigens are presented on the membrane of tumor cells and thus represent antigens on tumor cells, without however causing an immune reaction affecting the tumor disease of the patient. Rapp (U.S. Pat. No. 5,156,841) has already described the use of oncoproteins, i.e. expression products of the oncogenes, as an immunogen for tumor vaccines. Reference is explicitly made to this document.

Examples for cell antigens and their oncogenic mutations according to the invention are i) receptors, such as Her-2/neu, androgen receptor, estrogen receptor, midkine receptor, EGF receptor, ERBB2, ERBB4, TRAIL receptor, FAS, TNFalpha receptor; ii) signal-transducing proteins and their oncogenic mutations, such as c-Raf (Raf-1), A-Raf, B-Raf, Ras, Bcl-2, Bcl-X, Bcl-W, Bfl-1, Brag-1, Mcl-1, A1, Bax, BAD, Bak, Bcl-Xs, Bid, Bik, Hrk, Bcr/abl, Myb, C-Met, IAP1, IAO2, XIAP, ML-IAP LIVIN, survivin, APAF-1; iii) proteins of the cell cycle control and their oncogenic mutations, such as cyclin D(1-3), E, A, B, H, Cdk-1, -2, -4, -6, -7, Cdc25C, P16, p15, p21, p27, p18, pRb, p107, p130, E2F(1-5), GAAD45, MDM2, PCNA, ARF, PTEN, APC, BRCA, P53 and homologues; iv) transcription factors and their oncogenic mutations, such as C-Myc, NFkB, c-Jun, ATF-2, Sp1; v) embryonic proteins, such as carcinoembryonic antigen, alpha-fetoprotein, MAGE, PSCA; vi) differentiation antigens, such as MART, Gp100, tyrosinase, GRP, TCF-4; vii) viral antigens, such as of the following viruses: HPV, HCV, HPV, EBV, CMV, HSV.

Alternatively or additionally, component I) may represent at least one nucleotide sequence for at least one antigen that is specific for a normal tissue cell, from which the respective tumor originates. Such specific antigens are for instance i) receptors, such as androgen receptors, estrogen receptors, lactoferrin receptors; ii) differentiation antigens, such as basic myelin, alpha-lactalbumin, GFAP, PSA, fibrillary acid protein, tyrosinase, EGR-1, MUC1.

Component II).

Component II) represents at least one nucleotide sequence for at least one protein, which stimulates cells of the immune system. By the selection of the protein, the immune reaction to the expression product of component I) can be intensified and/or oriented more to the activation of Th1 cells (for the cellular immune reaction) or to the activation of Th2 cells (for the humoral immune reaction). Immune-stimulating proteins are for instance i) cytokines, such as M-CSF, GM-CSF, G-CSF; ii) interferons, such as IFN-alpha, beta, gamma; iii) interleukins, such as IL-1, -2, -3, -4, -5, -6, -7, -9, -10, -11, -12, -13, -14, -15, -16, human leukemia inhibitory factor (LIF), iv) chemokines, such as RANTES, monocyte chemotactic and activating factor (MCAF), macrophage inflammatory protein-1 (MIP-1-alpha, beta), neutrophil activating protein-2 (NAP-2), IL-8.

Component IIIA).

Component IIIA) is at least one nucleotide sequence coding for at least one transport system, which makes it possible to express the expression of the expression products of components I) and, optionally, II) on the outer surface of the microorganism. The respective component can as an option be either secreted or expressed on the membrane of the microorganism, i.e. is membrane-bound. Such transport systems are for instance i) the hemolysin transport signal of E. coli (nucleotide sequences containing HlyA, HlyB and HlyD under the control of the hly-specific promoter); the following transport signals are to be used: for the secretion—the C-terminal HlyA transport signal, in presence of HlyB and HlyD proteins; for the membrane-bound expression—the C-terminal HlyA transport signal, in presence of HlyB protein, ii) the hemolysin transport signal of E. coli (nucleotide sequences containing HlyA, HlyB and HlyD under the control of a not hly-specific bacterial promoter), iii) the transport signal for the Slayer protein (Rsa A) of Caulobacter crescentus; the following transport signals are to be used: for the secretion and the membrane-bound expression—the C-terminal RsaA transport signal, iv) the transport signal for the TolC protein Escherichia coli; the following transport signals are to be used: for the membrane-bound expression—the N-terminal transport signal of TolC (the integral membrane protein TolC of E. coli is a multi-functional pore-forming protein of the outer membrane of E. coli, which serves—in addition to functions such as the reception of colicin E1 (Morona et al., J. Bacteriol. 153:693-699, 1983) and the secretion of colicin V (Fath et al., J. Bacteriol. 173:7549-7556, 1991)—also as a receptor for the U3 phage (Austin et al., J. Bacteriol. 172:5312-5325, 1990); this protein is not only found in E. coli, but also in a multitude of gram-negative bacteria (Wiener, Structure Fold Des 8:R171-175, 2000); the localization in the outer membrane and the wide occurrence make TolC to an ideal candidate to present heterologous antigens, in order e.g. to cause an immune reaction.

Component IIIB).

Component IIIB) is a nucleotide sequence coding for at least one lytic protein, which is expressed in the cytosol of a mammalian cell and lyses the microorganism for releasing the plasmids in the cytosol of the host cell. Such lytic proteins (endolysins) are for instance Listeria-specific lysis proteins, such as PLY551 (Loessner et al Mol Microbiol 16:1231-41, 1995) and/or the Listeria-specific holin under the control of a listerial promoter.

A preferred embodiment of this invention is the combination of different components IIIB), for instance the combination of a lysis protein and the holin.

The components IIIA and/or IIIB may be constitutively active.

Component IV).

Component IV) represents at least one nucleotide sequence for at least one activation sequence for the expression of component I) and, optionally, II).

If the expression is membrane-bound on the outer surface of the microorganism, the activation sequence has preferably to be selected such that it is capable of being activated in the microorganism. Such activation sequences are for instance: i) constitutively active promoter regions, such as the promoter region with “ribosomal binding site” (RBS) of the beta-lactamase gene of E. coli or of the tetA gene (Busby and Ebright, Cell 79:743-746, 1994); ii) promoters, which are capable of being induced, preferably promoters, which become active after reception in the cell. To these belong the actA promoter of L. monocytogenes (Dietrich et al., Nat. Biotechnol. 16:181-185, 1998) or the pagC promoter of S. typhimurium (Bumann, Infect Immun 69:7493-7500, 2001).

If the plasmids are released from the microorganism after its lysis into the cytosol of the cell, the activation sequence is not cell-specific, but tissue cell-specific, cell cycle-specific or function-specific. Preferably, such activation sequences are selected, which are particularly activated in macrophages, dendritic cells and lymphocytes.

Microorganisms in the meaning of the invention are viruses, bacteria or unicellular parasites, which are usually used for the transfer of nucleotide sequences being foreign for the microorganism.

In a special embodiment of this invention, the microorganisms represent gram-positive or gram-negative bacteria, preferably bacteria, such as Escherichia coli, Salmonella, Yersinia enterocolitica, Vibrio cholerae, Listeria monocytogenes, Shigella.

Preferably, such bacteria are used, which are attenuated in their virulence.

The components according to the invention are introduced into the microorganisms by methods well known to the man skilled in the art. If the microorganisms represent bacteria, the components are inserted into plasmids, and the plasmids are transferred into the bacteria. The techniques suitable for this and the plasmids are sufficiently known to the man skilled in the art.

Subject matter of the invention are medicament preparations containing the microorganisms according to the invention or however membrane envelopes of these microorganisms. The preparation of these membrane envelopes takes for instance place according to the method described in EP-A-0,540 525. Such medicament preparations are for instance suspensions of the microorganisms according to the invention in the solutions familiar to the pharmacist, suitable for injection.

Another subject matter of the invention is the administration of a medicament preparation containing the microorganisms according to the invention. The administration is made locally or systemically, for instance into the epidermis, into the subcutis, into the musculature, into a body cavity, into an organ, into the tumor or into the blood circulation.

A particular subject matter of this invention is the peroral or rectal administration of the medicament according to the invention for the prophylaxis and/or therapy of a proliferative disease. The administration can be made once or several times. In each administration, approximately 10 to 10ˆ9 microorganisms according to the invention are administered. If the administration of this number of microorganisms according to the invention does not cause a sufficient immune reaction, the number to be injected has to be increased.

After administration of the microorganisms according to the invention, the tolerance for a cell presenting component I), for instance for a tumor cell, or for a tissue cell, from which the tumor originates, is broken, and a cytotoxic immune reaction directed against the tumor and/or its tissue cells is triggered.

Depending on the selection of component I), this cytotoxic immune reaction is directed either exclusively against the tumor or also against the tumor cells including the tissue cells, from which the tumor cells originate.

Subject matter of the invention is thus the administration of a medicament preparation according to the invention for the prophylaxis or therapy of a proliferative disease. Proliferative diseases are tumor diseases, leukemias, virally caused diseases, chronic inflammations, rejections of transplanted organs and autoimmune diseases.

In a special embodiment of this invention, wherein component I) represents at least one cell antigen, which is expressed by a tumor cell and the tissue cells, from which the tumor originates, the medicament according to the invention is administered for the prophylaxis or therapy of a tumor of the glandula thyroidea, the mamma, the stomach, the kidney, the ovarium, the nevi, the prostate, the cervix or the vesica urinaria.

In the following, the invention is explained in more detail, based on examples representing embodiments only.

### EXAMPLE 1

### Induction of an Immune Response in BxB Mice by Immunization with Salmonellae Expressing c-raf

Raf is a normally cytosolic serine/threonine kinase (PSK), which in conjunction with other proteins of signal cascades controls the cell growth and survival (Kerkhoff and Rapp, Oncogene 17:1457-1462, 1998; Troppmair and Rapp, Recent Results Cancer Res. 143:245-249, 1997). A binding of a growth factor to a respective receptor normally leads via an activation of Ras, the subsequent activation of Raf via several phosphorylation steps via the PSK and tyrosine kinase MEK and the PSK ERK to an activation of the replication machinery in the cell nucleus (Kerkhoff and Rapp, Oncogene 17:1457-1462, 1998). The first link in this chain, the small G protein Ras, is present in a modified form in 30% of all human tumors (Zachos and Spandidos, Crit. Rev. Oncol. Hematol. 26:65-75, 1997). Raf is an effector of Ras and is present in an over-expressed form in a multitude of human tumors (Naumann et al., Recent Results Cancer Res. 143:237-244, 1997).

For the test in the mouse model, transgenic mice were used, which over-express the complete molecule or the constitutively active kinase domain (BxB) (Kerkhoff et al., Cell Growth Differ 11:185-190, 2000). Therewith, the mice spontaneously develop lung tumors approx. half a year later.

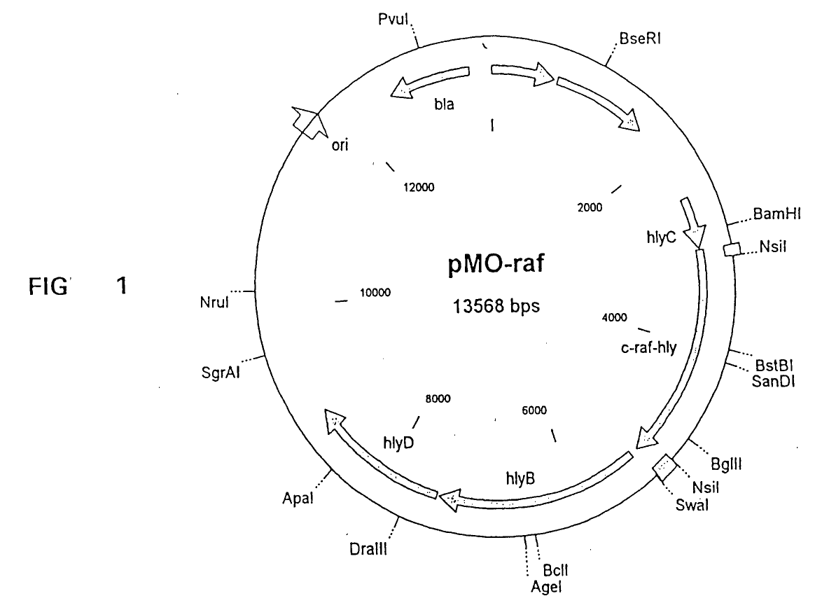

For the generation of the vaccines, the human c-Raf cDNA was cloned by means of PCR in-frame with HlyA into the plasmid pMOhly 1 (FIG. 1). Subsequently, the plasmid pMO-Raf was transfected into attenuated salmonellae (S. typhi murium SL7207), which carry a defect in the aromatic metabolism (Hoiseth and Stocker, Nature 291:238-239, 1981). In the immune blotting by means of antibodies directed against c-Raf, the c-Raf HlyAs fusion protein could be detected in the bacterium lysate as well as in the culture supernatant of SL7207 bacteria transfected with PMOhy-Raf.

BxB transgenic mice were orally immunized at an age of 7-10 weeks with the salmonellae (dose 5×10ˆ9), and the vaccination was repeated twice in an interval of 5 days. 45 days after the last immunization, an intravenous refreshing vaccination with 5×10ˆ5 salmonellae was made. For control purposes, naked c-Raf coding DNA was intramuscularly administered to the mice.

5-7 days after the last immunization, now serum samples were taken, and the antibody response was analyzed by means of a Western blot. For this purpose, the 1:200 diluted serum was hybridized against membranes with separated protein and blotted protein of c-Raf-transfected or not transfected bacteria. The detection of the bound serum antibodies took place by means of antibodies specific for mouse IgG. In contrast to the control mice, immunized with pMohly-Raf transfected SL7207, c-Raf-specific antibodies of the isotype IgG could be induced. Thus it has been shown that an immunization with the described salmonellae can break the self-tolerance and induces CD4+ T cells, which are necessary for the antibody isotype change to IgG.

For the analysis of the CD8+ T cell response, C57BL-6 mice were immunized following the same protocol. 7 days after the last immunization, spleen cells were isolated, and they were stimulated with Raf-over-expressing EL-4 cells. 1 h after beginning the stimulation, the vesicular transport was blocked by Brefeldin A, and after another 4 h, the cells were stained with CD8 and IFN-g-specific antibodies and analyzed by flow cytometry (Mittrucker et al., Infect Immun 70:199-203, 2002). Only in one pMO-Raf-immunized mouse, a Raf-specific antibody response could be detected.

For detecting the tumoricidal activity, 10, 12 and 14 months old immunized and not immunized BxB mice were killed, and the lung mass was weighed. The lung mass is a direct measure for the size of the tumor. In the group, immunized with SL-pMO-Raf, after 14 months clearly more frequently mice with a reduced lung mass could be found than in the control groups including the group, which has been immunized with naked DNA coding for c-Raf (SL-pCMV-raf). Normally, the tumor growth on not treated animals is not reversible (Kerkhoff et al., Cell Growth Differ. 11:185-190, 2000). These data thus show that in this experiment a vaccination with SL-pMO-Raf animals could protect from the generation of tumors, and the invention described here is suitable as a tumor vaccine.

These experiments further show that the carrier system represented in this invention can in principle break the self-tolerance and induce in c-Raf-tolerant animals a c-Raf-specific antibody response and T cell response.

By means of the same experimental systems, salmonellae can be produced as vaccines, which express isoforms of C-Raf (such as for instance B-Raf and A-Raf), mutated C-Raf, B-Raf or A-Raf, epitopes of normal or mutated C-Raf, B-Raf or A Raf, or combinations of epitopes of normal and/or mutated C-Raf, B-Raf or A-Raf. Examples for a mutation coming along with a loss of the activity of Raf are mutations of the Ras-binding domain, the kinase domain and/or the phosphorylation sites.

### EXAMPLE 2

### Induction of an Immune Response in BALB/C Mice by Immunization with Salmonellae Expressing PSA

The existence of tissue-specific antigens, in particular of those, which are synthesized and expressed to a high degree by tumor cells, is, beside the diagnostic usability of these markers, also a possible starting point for therapeutic approaches. For the prostate carcinoma, up to now three antigens worth mentioning have been identified: PSA (prostate-specific antigen), PSMA (prostate-specific membrane antigen) and PSCA (prostate stem cell antigen). Whilst PSA exists already in early tumor forms in an over-expressed manner (Watt et al., Proc. Natl. Acad. Sci. USA 83:3166-3170, 1986; Wang et al., Prostate 2:89-96, 1981) and thus contributes for carcinoma diagnosis (Labrie et al., J. Urol. 147:846-851; discussion 851-842, 1992), the PSCA expression is in most cases only increased in the locally advanced, dedifferentiated and metastasized tumor stage (Gu et al., Oncogene 19:1288-1296, 2000; Reiter et al., Proc. Natl. Acad. Sci. USA 95:1735-1740, 1998). The organ specificity makes PSA as well as PSCA to a potential target antigen for the development of immune therapies against the prostate carcinoma (Reiter et al., Proc. Natl. Acad. Sci. USA 95:1735-1740, 1998; Hodge et al., Int. J. Cancer 63: 231-237, 1995; Armbruster, Clin. Chem. 39:181-195, 1993).

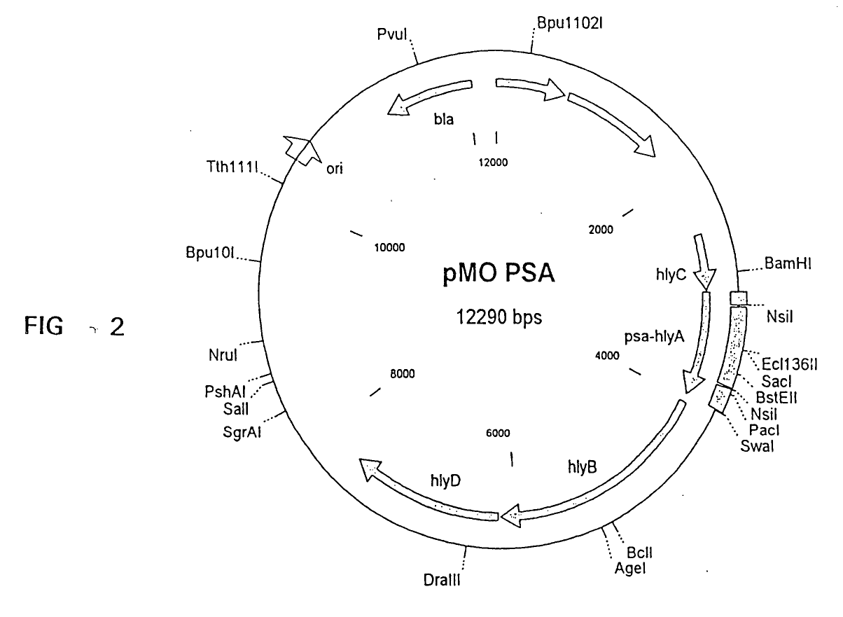

In this first experiment, it was intended to show whether PSA-secerning salmonellae on the base of the vector pMOHLY 1 can induce an immune response in BALB/c mice. For this purpose, first two NsiI interfaces were introduced by polymerase chain reaction (PCR) into the c-DNA sequence of PSA, in order to make an in-frame insertion of the amplified fragment into the target vector possible. For the amplification, a fragment of 645 base pairs (bp) was selected. As primers served 5′-GTGGATTGGTGATGCATCCCTCATC-3′ and 5′-CAGGGCACATGCATCACTGCCCCA-3′. The PCR product was first cloned blunt-end into the vector pUC18 and later ligated via NsiI interfaces with the target vector pMOhlyl. The correct insertion was controlled by means of restriction digestion and confirmed by sequentiation (FIG. 2).

By means of this salmonella strain, BALB/c mice were now nasally immunized three times in an interval of 3 weeks with a dose of 1×10⁷. The immune response is detected with Western blot analyses and intracellular cytokine staining.

1. A microorganism with a nucleotide sequence coding for a cell antigen, in the genome of which the following components are inserted and are expressible:
I) a nucleotide sequence coding for at least one epitope of an antigen or several antigens of a tumor cell or a nucleotide sequence for at least one epitope of an antigen or several antigens that is or are specific for a tissue cell from which the tumor originates;
II) a nucleotide sequence coding for a protein that stimulates cells of the immune system;
IIIA) a nucleotide sequence for a transport system, which makes it possible to express the expression product of components I)
IIIB) a nucleotide sequence for a protein for lysing the microorganisms in the cytosol of mammalian cells and for intracellularly releasing plasmids, which are contained in the lysed microorganisms; and
IV) an activation sequence for expressing one or several of components 1) to IIIB), said activation sequence being selected from the group consisting of an activation sequence, which is capable of being activated in the microorganism, or which is tissue-cell-specific, or which is not cell-specific,
wherein each of components 1) to IV) can be identical or different, and each present once or multiple.

2. The microorganism according to claim 1, wherein the microorganism is a virus or a bacterium comprising a gram-positive or gram-negative bacterium, further comprising Escherichia coli, Salmonella, Yersinia enterocolitica, Vibrio cholerae, Listeria monocytogenes, and Shigella, or is a unicellular parasite, the virulence of the microorganism being reduced.

3. The microorganism according to claim 1, wherein the microorganism is the envelope of a bacterium.

4. The microorganism according to claim 1, wherein component I) is a nucleotide sequence coding for an epitope or several epitopes of an antigen or several antigens of a protein or several proteins of a tumor cell, wherein this protein comprises extracellular, transmembranic or intracellular part of a receptor; extracellular, transmembranic or intracellular part of an adhesion molecule; signal-transducing protein; a protein controlling the cell cycle; transcription factor; differentiation protein; embryonic protein; and viral protein, wherein the protein is an oncogenic gene product or a suppressor gene product comprising c-raf, A-Raf, B-Raf or a homologous protein of c-Raf, A-Raf or B-Raf.

5. The microorganism according to claim 1, wherein component I) is a nucleotide sequence coding for an antigen that is specific for the tissue cell comprising glandula thyroidea, glandula mammaria, glandula salivaria, nodus lymphoideus, glandula mammaria, tunica mucosa gastris, kidney, ovarium, prostate, cervix, tunica serosa vesicae urinariae and nevus, from which the tumor originates.

6. The microorganism according to claim 1, comprising a component I) according to claim 4 and a component I) according to claim 5.

7. The microorganism according to claim 1, wherein component II) codes for at least one cytokine, interleukin, interferon or chemokine.

8. The microorganism according to claim 1, wherein component IIIA) codes for the hemolysin transport signal of Escherichia coli, the Slayer (Rsa A) protein of Caulobacter crescentus or for the TolC protein of Escherichia coli.

9. The microorganism according to claim 1, wherein component IB) codes for a lytic protein of gram-positive bacteria, a lytic protein of Listeria monocytogenes, for PLY551 of Listeria monocytogenes or the holin of Listeria monocytogenes.

10. The microorganism according to claim 1, wherein component IV) codes for an activator sequence capable of being activated in the microorganism comprising a tumor cell-specific, tissue cell-specific, macrophagespecific, dendrite-specific, lymphocyte-specific, function-specific activator sequence or an activator sequence being cell-non-specifically activated.

11. The microorganism according to claim 1, wherein component I) codes for at least two different proteins.

12. A method for the prophylaxis or therapy of a disease, which is caused by uncontrolled cell division or an infection comprising a tumor disease, further comprising a prostate carcinoma, an ovary carcinoma, a mamma carcinoma, a stomach carcinoma, a kidney tumor, a tumor of glandula thyroidea, a melanoma, a tumor of cervix, a tumor of vesica urinaria, a tumor of glandula salivaria or a tumor of nodus lymphoideus, a leukemia, a viral or bacterial infection, a chronic inflammation, an organ rejection or an autoimmune disease comprising administering a physiologically effective dose of a medicament comprising a microorganism according to claim 1.

13. The method according to claim 12 further comprising the removal of a tumor as well as of the healthy tissue from which the tumor originates.

14. The method according to claim 12, wherein the medicament is prepared for local, parenteral, oral or rectal administration.

15. A method for the production of a medicament according to claim 12, wherein a microorganism according to claim 1 is prepared in a physiologically effective dose with one or several physiologically tolerated carrier substances for oral, intramuscular, intravenous, intraperitoneal, rectal or local administration.

16. A plasmid or expression vector comprising the components I) to IV) according to claim 1.

17. A method for the production of a microorganism according to claim 1, wherein a plasmid or expression vector according to claim 16 is produced, and a microorganism is transformed with this plasmid or expression vector.

18. The microorganism of claim 1, wherein the nucleotide sequence of component IIIA) is capable of causing the expression of the expression product of component I) on the outer surface of the bacterium, or secretion of the expression product of component I).

19. The microorganism of claim 1, wherein the nucleotide sequence of component IIIA) is capable of causing the expression of the expression product of component II).
